(19) United States
(12) Patent Application Publication
(10) Pub. No.: US 2024/0408025 A1
(43) Pub. Date: Dec. 12, 2024

(54) A FILM-FORMING COMPOSITION COMPRISING PECTIN

(71) Applicants: NUTRITION & BIOSCIENCES USA 1, LLC; INTERNATIONAL N&H USA, INC., Wilmington, DE (US)

(72) Inventors: Rebecca Putans, Midland, MI (US); Benjamin Roscoe, Wilmington, DE (US); Michael Baumann, Walsrode (DE)

(21) Appl. No.: 18/691,680

(22) PCT Filed: Aug. 18, 2022
(86) PCT No.: PCT/EP2022/073076

§ 371 Date: Mar. 13, 2024

Related U.S. Application Data
(60) Provisional application No. 63/243,478, filed on Sep. 13, 2021.

(30) Foreign Application Priority Data
Nov. 23, 2021 (EP) 21209774.5

(51) Int. Cl. A61K 9/48; A61K 9/28; A61K 45/06

(52) U.S. Cl. CPC A61K 9/4816; A61K 9/286; A61K 9/2893; A61K 45/06

(57) ABSTRACT

The present specification relates to a film-forming composition comprising high ester (HE) pectin as a film-forming polymer, water and optionally a plasticizer. This specification further relates to capsules comprising soft or hard capsule shells made from said film-forming composition.

### FIELD

The present specification relates to a film-forming composition comprising a pectin as a film-forming polymer as well as the use of the film-forming composition for the preparation of capsules and coatings.

### BACKGROUND

Capsules are the second most common solid oral dosage form after traditional tablets. As of 2014 the estimated market size for capsules was $2 billion, which can be further segmented into hard capsules and soft capsules. Both the hard and soft capsule market are currently dominated by gelatin which is a hydrolyzed protein derived from collagen obtained by boiling animal bones and cartilage in water under pressure. However due to consumer preference for health, dietary, or religious reasons, the use of gelatin is declining and other materials continue to make headway. Specifically in soft capsules, vegetable materials such as carrageenan (e.g. SeaGel® available from IFF), have been able to capture about 13% of the soft capsule market, with an expected growth rate of 14%.

Carrageenan based soft-capsule systems have been on the market for several years, however in that time there has been consumer hesitance to use the products due to a misperception of carrageenan safety. There have been some scientific reports and consumer group reports that erroneously attributed negative toxicological effects in animals to carrageenan, when the material that was actually tested was a degraded carrageenan or poligeenan. There has been confusion within the scientific community and the public between carrageenan that is used as a food additive, with molecular weight between 200-800 kDa, and degraded forms of carrageenan such as d-CGN (MW range of 20-40 kDa), and PGN (MW range of 10-20 kDa), cf. McKim J M, Willoughby J A Sr, Blakemore W R, Weiner M L: Clarifying the confusion between poligeenan, degraded carrageenan, and carrageenan: A review of the chemistry, nomenclature, and in vivo toxicology by the oral route. Crit Rev Food Sci Nutr. 2018 Jun. 14:1-20; James M. McKim, Jamin A. Willoughby Sr., William R. Blakemore, and Myra L. Weiner: “Clarifying the confusion between poligeenan, degraded carrageenan, and carrageenan: A review of the chemistry, nomenclature, and in vivo toxicology by the oral route”. Crit Rev Food Sci Nutr. 2019, VOL. 59, NO. 19, 3054-3073; Myra L. Weiner, James M. McKim, Jr.: Comment on “Revisiting the carrageenan controversy: do we really understand the digestive fate and safety of carrageenan in our foods?” by S. David, C. S. Levi, L. Fahoum, Y. Ungar, E. G. Meyron-Holtz, A. Shpigelman and U. Lesmes, Food Funct., 2018, 9, 1344-1352; James M. McKim Jr., Heidi Baas, Gabriel P. Rice, Jamin A. Willoughby Sr., Myra L. Weiner, William Blakemore: Effects of carrageenan on cell permeability, cytotoxicity, and cytokine gene expression in human intestinal and hepatic cell lines. Food and Chemical Toxicology, Volume 96, October 2016, Pages 1-10; Myra L. Weiner: Parameters and pitfalls to consider in the conduct of food additive research, Carrageenan as a case study. Food and Chemical Toxicology, Volume 87, January 2016, Pages 31-44; Myra L. Weiner, James M. McKim, William R. Blakemore: Addendum to Weiner, M. L. (2016) Parameters and Pitfalls to Consider in the Conduct of Food Additive Research, Carrageenan as a Case Study. Food and Chemical Toxicology, Volume 107, Part A, September 2017, Pages 208-214.

Due to the perceived issue with using carrageenans in capsule systems, there is a need in the art for identifying alternative non-gelatin materials that have properties that make them suitable for capsule materials. An alternative vegetable material that can be included in a film-forming composition is pectin which has been disclosed in the art as a film-forming polymer for the preparation of hard or soft capsules, cf. for instance WO 02/17886. The pectin disclosed for this purpose is low ester (LE) pectin which confers enteric properties to capsules such that they are insoluble under gastric conditions (pH 1.2) and readily soluble under intestinal conditions (pH 6.8). The pectin is typically combined with a setting system in the composition of WO 02/17886. At least one other film-forming polymer is required to improve the mechanical performance of the capsules.

### SUMMARY

An object of the present specification is to provide a suitable non-animal (gelatin-free) replacement for carrageenan in soft and hard capsules intended for immediate release of active ingredients incorporated in the capsules. Several candidates were tested based on physical properties, such as film forming ability, temperature processing windows, material origin and material cost. A favorable candidate as a film-forming polymer that provides for immediate release of active ingredients in capsules or tablets was found to be high ester (HE) pectin.

Accordingly, the present specification relates to a film-forming composition comprising high ester (HE) pectin as a film-forming agent, water and optionally a plasticizer.

In another aspect, this specification relates to a capsule comprising a capsule shell prepared from said film-forming composition and a fill material.

In a further aspect, this specification relates to the use of said film-forming composition to provide a coating on a solid dosage form.

### BRIEF DESCRIPTION OF THE DRAWINGS

FIG. 1 is a graph showing the puncture strength of films made from HE pectins with or without buffering agent and with or without HP starch and with varying amounts of glycerine as plasticizer, compared to films containing carrageenan.

FIG. 2 is a graph showing the elasticity of films made from HE pectins with or without buffering agent and with or without HP starch and with varying amounts of glycerine as plasticizer, compared to films containing carrageenan.

FIG. 3 is a graph showing the hardness, burst strength and distance to burst of capsules prepared in Example 3 compared to capsules containing carrageenan.

FIG. 4 is a graph showing the release of theophylline from uncoated tablets and tablets coated with the film-forming composition comprising 3% HE pectin.

### DETAILED DESCRIPTION

Pectin is a structural polysaccharide typically found in the form of a water insoluble parent pectic substance—protopectin—in the primary cell wall and the middle lamella of green land plants such as fruit and vegetables. Major sources of commercial pectin products are citrus peel and apple pomace in which protopectin represents 10-40% by weight of the dry matter.

Pectin is the generic designation for water-soluble compounds which result from restricted hydrolysis of protopectin. The exact nature of protopectin is not completely understood. It is, however, generally recognized that protopectin is a complex structure in which pectin is attached to other cell wall components such as cellulose, cell wall protein and hemicellulose by covalent bonds, hydrogen bonds and/or ionic interactions.

Pectin comprises linear galacturonan chains (polymer of α-(1-4)-linked-D-galacturonic acid) which are interrupted with rhamno-galacturonan backbones (polymers of the repeating disaccharide α-(1-4)-D-galacturonic acid-α-(1-2)-L-rhamnose), which often has side chains of polymeric arabinogalactans glycosidic linked to the O-3 or O-4 positions of L-rhamnose. The galacturonan sequences can have D-xylose and D-apiose glycosidic linked to their O-2 or O-3 positions, which also can be substituted with ester-linked acetyl groups. The long chains of α-(1-4)-linked D-galacturonic acid residues are commonly referred to as “smooth regions”, whereas the highly branched rhamnogalacturonan regions are commonly referred to as the “hairy regions”.

Pectin molecules have a molecular weight of up to more than 200,000 Da and a degree of polymerization up to more than 1000 units. A proportion of the carboxylic acid groups of the galacturonic acid units are methyl-esterified. In plants the residual carboxyl groups are partly or completely neutralized with cations of calcium, potassium and magnesium which inherently are contained in the plant tissues.

The “degree of esterification” (DE) means the extent to which free carboxylic acid groups contained in the galacturonic acid units of pectin have been methyl esterified. The resultant pectin is referred to as “high ester pectin” (“HE pectin” for short) if more than 50% of the carboxyl groups are esterified. The resultant pectin is referred to as a “low ester pectin” (“LE pectin” for short or a “low methoxyl pectin”) if less than 50% of the carboxyl groups are esterified. Pectin is also available in amidated form wherein 8-25% of the carboxyl groups are substituted with amide groups.

The structure of pectin, in particular the degree of esterification, determines its physical and/or chemical properties. For example, pectin gelation depends on the chemical nature of pectin, especially the degree of esterification and degree of polymerization. In addition, however, pectin gelation also depends on the pectin concentration and environmental conditions like soluble solids content, the pH and calcium ion concentration.

In the film-forming composition in this specification, favorable results are obtained in terms of film-forming properties and immediate release when the HE pectin exhibits a degree of esterification higher than 52%, such as higher than 54, such as higher than 56, or higher than 58%, such as of about 60%. In the present context, “immediate release” is intended to indicate that an active ingredient/substance is released within 15 minutes from immersion in simulated gastric fluid at pH 1.2. It is generally preferred that the HE pectin exhibits a degree of amidation of 0% although minor quantities of amidated pectin may be added to the composition if delayed release properties are desired in the final film. The composition of this specification may favorably comprise HE pectin in an amount of 10-35% by weight of the composition.

In an embodiment, the film-forming composition of this specification may further comprise a second film-forming agent to mitigate the brittleness of films made from HE pectin alone which may result in the composition not filling the die cavities in the rotary die encapsulation process and consequently lead to underfilled capsules. Examples of suitable second film-forming agents are a starch, starch hydrolysate, starch derivative, β-1,3-glucan, cellulose gum, hydrocolloid such as xanthan gum, alginate, carrageenan, or an alkylcellulose ether such as methylcellulose or hydroxypropyl methylcellulose. The second film-forming agent may contribute to favorable film-forming properties to the composition such as increased puncture strength and elasticity.

In a preferred embodiment, the composition of this specification may include a native or chemically modified starch as the second film-forming agent. In this context, “native starch” is intended to mean starch that has either not been subjected to chemical modification and/or been only minimally processed. “Chemically modified starch” is intended to mean starch derivatives subjected to chemical substitution such as hydroxypropylation, carboxymethylation or other etherification step. The starch may be selected from the group consisting of corn starch, rice starch, potato starch, pea starch, tapioca starch, wheat starch, rye starch, oat starch, barley starch or starch derived from pulses, and mixtures thereof. It is generally preferred that the starch contains less than 100% amylopectin, i.e. that it is not a waxy starch. A currently favored chemically modified starch may for instance be hydroxypropylated starch or hydroxyethylated starch, preferably hydroxypropylated starch.

In another embodiment, the present film-forming composition may comprise a plasticizer in order to mitigate the brittleness and improve the flexibility of films made from HE alone. In this embodiment, the plasticizer may be selected from the group consisting of glycerine, sorbitol, lactitol, maltitol, polydextrose, polyethylene glycol or mixtures thereof.

In an embodiment the film-forming composition of this specification further comprises a divalent cation salt. Such a salt has been found to facilitate setting of the HE pectin when the film-forming composition is used to prepare capsules by dipping. It has in particular been found that the divalent cation salt is effective when it is a poorly water-soluble calcium salt such as CaCO₃.

In a further embodiment, the film-forming composition further comprises one or more buffering agents which may, for instance, be selected from the group consisting of sodium citrate, sodium potassium tartrate and sodium tripolyphosphate.

In a further embodiment, this specification relates to a capsule comprising a capsule shell prepared from the film-forming composition of this specification and a fill material. The fill material within the capsule shell may typically comprise a pharmaceutically active ingredient, dietary supplement, cosmetic, flavouring agent, foodstuff, agrochemical or scent.

In an embodiment, the capsule may comprise a hard capsule shell. Hard capsule shells are generally manufactured by using a dip molding process. In this process, pin molds are dipped into a film forming composition. By gelling the film forming polymer on the pin, a film is formed that is subsequently dried on the pin to obtain a capsule shell. The shells are then stripped off the pins and cut to a desired length. Thus, capsules caps and bodies are obtained that can later be filled with a substance and joined such that a filled capsule is obtained. When using this type of dip molding process, it is necessary to ensure that the dipping composition adheres to the pin surface and quickly gels, once the pins are withdrawn from the dipping bath. This keeps the composition from flowing on the surface of the pins so as to achieve the desired shell or film thickness distribution to manufacture capsules.

In an alternative embodiment, the capsule may comprise a soft capsule shell. Soft capsule shells may typically be prepared by the rotary die process where two film ribbons are each cast on a rotating drum, passed under a heated wedge, and are then subsequently pressed between two dies that have the desired size/shape cavity. The capsules are filled through the wedge as they are being pressed into the die pockets, essentially as described in U.S. Pat. No. 6,340,473 B1, WO 98/42294 and The Theory and Practice of Industrial Pharmacy, Lachman, Lieberman and Kanig, Eds, 3^{rd} Ed.

In particular when preparing soft capsules, the fill material may be a liquid or semi-solid material that includes a pharmaceutically active ingredient, dietary supplement, cosmetic, flavouring agent, foodstuff, agrochemical or scent.

Both hard and soft capsules of this specification provide for immediate release of the active ingredient in the fill material.

The capsule shell may further comprise optional additives, such as coloring agents, flavor and taste improvers, antioxidants, plasticizers, bulking agents and surfactants. For example, when producing capsules a water-soluble food dye, such as red oxide, or a natural dye, may be used as a coloring agent; TiO₂ may be used as a opacifying agent.

In a further aspect of the present specification, the present film-forming composition may be used for coating dosage forms, such as tablets, granules, pellets, caplets, lozenges, suppositories, pessaries or implantable dosage forms, to form a coated composition providing immediate release of an active ingredient contained in the dosage form. Preferred dosage forms are pharmaceutical dosage forms, nutrition supplements or agricultural dosage forms.

Useful additives for coatings of solid forms are plasticizers, solids-loading enhancers, a second film-forming polymer, surfactants, lubricants, polishing agents, pigments, anti-tack agents, glidants, opacifiers, coloring agents and any combination thereof.

Numbered embodiments of the present specification:
1. A film-forming composition comprising high ester (HE) pectin as a film-forming polymer, water and optionally a plasticizer.
2. The composition of embodiment 1, wherein the HE pectin exhibits a degree of esterification higher than 52%, such as higher than 54, such as higher than 56, or higher than 58%, such as of about 60%, such as in the range of 52%-60%, such as in the range of 54%-60%, such as in the range of 56%-60%, such as in the range of 58%-60%.
3. The composition of embodiment 1 or 2, wherein the HE pectin exhibits a degree of amidation of 0%.
4. The composition of any one of embodiments 1-3 comprising HE pectin in an amount of 10-35% by weight of the composition.
5. The composition according to any one of embodiments 1˜4 further comprising one or more buffering agents, for instance selected from the group consisting of sodium citrate, sodium potassium tartrate and sodium tripolyphosphate.
6. The composition of any one of embodiments 1-5 further comprising a second film-forming polymer selected from the group consisting of a native or chemically modified starch, xanthan gum, sodium alginate, β-1,3-glucan and carrageenan.
7. The composition of embodiment 6, wherein the starch is selected from the group consisting of corn starch, rice starch, potato starch, pea starch, tapioca starch wheat starch, rye starch, oat starch, barley starch and starch from pulses, and mixtures thereof.
8. The composition of embodiment 6 or 7, wherein the starch contains less than 100% amylopectin.
9. The composition of embodiment 6 or 7, wherein the starch is hydroxypropylated starch or hydroxyethylated starch, preferably hydroxypropylated starch.
10. The composition of any one of embodiments 1-9, wherein the plasticizer is selected from the group consisting of glycerol, sorbitol, maltitol, polydextrose, polyethylene glycol or mixtures thereof.
11. The composition of any one of embodiments 1-10 further comprising a divalent cation salt.
12. The composition of embodiment 11, wherein the divalent cation salt is a poorly water-soluble calcium salt such as CaCO₃.
13. A capsule comprising a capsule shell prepared from the film-forming composition of any one of embodiments 1-12 and a fill material.
14. The capsule of embodiment 13 comprising a soft capsule shell.
15. The capsule of embodiment 13 comprising a hard capsule shell.
16. The capsule of any one of embodiments 13-15, wherein the fill material comprises a pharmaceutically active ingredient, dietary supplement, flavouring agent, foodstuff, agrochemical or scent.
17. The capsule of embodiment 13, wherein the fill material is a liquid, semi-solid, or solid material.
18. The capsule of any one of embodiments 13-17 for immediate release of a pharmaceutically active ingredient, dietary supplement, flavouring agent, foodstuff, agrochemical or scent included in the fill material.
19. Use of a film-forming composition of any one of embodiments 1-12 to provide a coating on a solid dosage form.
20. The use of embodiment 19, wherein the coating is an immediate release coating.

### EXAMPLES

### Example 1

Film made from compositions comprising HE pectin as the sole film-forming polymer

Film forming compositions were prepared
from the following ingredients.

| Ingredient | Example 1a | Example 1b | Example 1c |
|---|---|---|---|
| HE pectin | 35% | 20% | 20% |
| Glycerine | 25% | 30% | 40% |
| Water | 40% | 50% | 40% |

Gel masses were prepared in a Premiere Mill (Netzsch) bench top mixer equipped with two anchor blades. Water and glycerine were weighed and charged into a jacketed mixing bowl. The bowl was then heated via an external temperature bath as the solid ingredients were weighed. HE pectin was weighed into a 1-gallon Ziploc bag and was homogenized by hand to ensure no agglomeration. Once the liquid ingredients reached 50-55° C., HE pectin was charged into mixing bowl. The gel was mixed for at least 2 hours and brought to an internal temperature of 90-95° C. The prepared gel mass was evaluated on visual appearance and ability to form films.

Wet film puncture strength and elasticity were measured using a TA.XT plus texture analyzer (available from Stable Microsystems). A 6.35 mm round probe travelling at 1 mm per second punctured the films and the maximum force and distance to puncture were recorded.

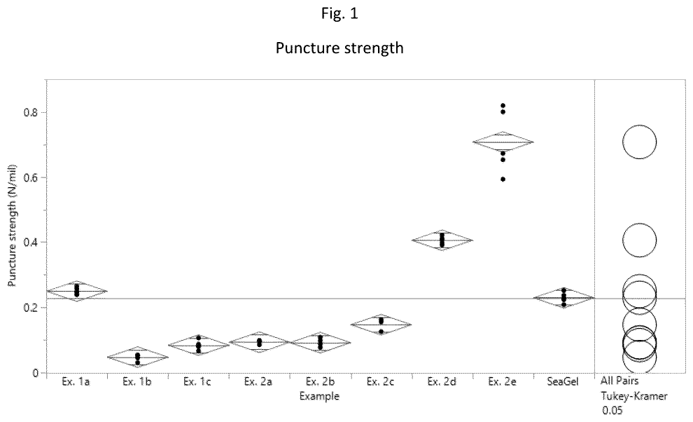

HE pectin films showed good film quality, in terms of clarity and feel. FIG. 1 shows the resulting puncture strength of the films. Puncture strength increased as the pectin concentration increased, and pectin films were able to match SeaGel® (kappa-2-carrageenan) puncture strength). Example 1a blend with 35% HE pectin showed the highest puncture strength, which was likely due to a higher polymer concentration.

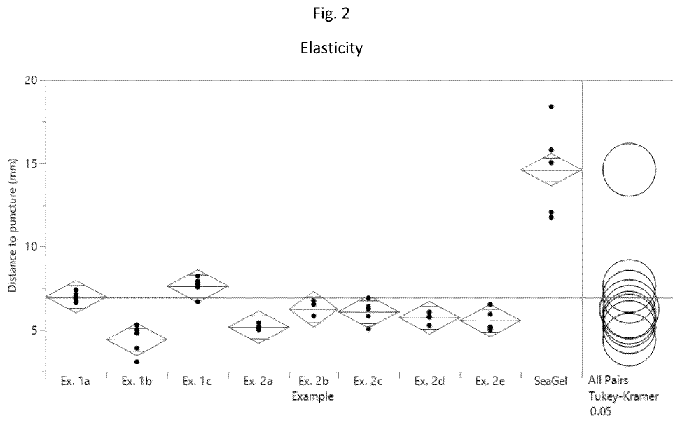

FIG. 1 shows the puncture strength. FIG. 2 shows the elasticity profile of films made from HE pectin and increasing levels of plasticizer compared to a film made from kappa-2-carrageenan (SeaGel®). It appears from FIG. 2 that increasing the level of plasticizer did not increase the elasticity of the HE pectin films.

### Example 2

Films Made with HE Pectin and HP Starch as the Second Film-Forming Polymer

Film-forming compositions were prepared
from the following ingredients.

| Ingredients | Ex. 2a | Ex. 2b | Ex. 2c | Ex. 2d | Ex. 2e |
|---|---|---|---|---|---|
| HE pectin | 10% | 15% | 20% | 10% | 10% |
| HE pectin cont. | 0% | 0% | 0% | 1% | 10% |
| buffering agents | | | | | |
| HP starch | 25% | 20% | 15% | 24% | 15% |
| Glycerine | 25% | 25% | 25% | 25% | 25% |
| Water | 40% | 40% | 40% | 40% | 40% |

To prepare the film composition of Example 2c, 250 g of glycerine and 400 g of water were added to a Premiere Mill mixing apparatus and were heated with a recirculating oil bath until they reached ˜50-60° C. While the liquid components were mixing, 200 g of HE pectin and 150 g of HP modified starch were weighed into a sealable plastic bag until uniform. The solid ingredients were then charged into the mixing bowl while stirring. The ingredients were mixed for 2 hours, reaching a final temperature of ˜93° C. Films were cast by hand using a 1.27 mm draw down knife onto a Teflon sheet. Films were left in ambient lab conditions overnight to dry.

Additional gels were prepared with blends of HE pectin and HE pectin containing buffering agents as shown in the table above. The blend samples showed improved mechanical properties, cf. FIGS. 1 and 2.

### Comparative Example A

Gels were prepared as in example 2, however an amidated pectin (24% esterification, 23% amidation) was used in place of the HE pectin.

### Comparative Example B

200 g of glycerine and 400 g of water were added to a Premiere Mill mixing apparatus and were heated with a recirculating oil bath until they reached 40° C. Then, 400 g of 225 bloom gelatin was charged into the liquid ingredients and vacuum was applied to the mixture. The ingredients were mixed for 2 hours reaching a final temperature of ˜70° C.

Disintegration measurements were performed in pH 1.2, pH 6.8, and pH 12 buffer solutions using a standard pharmaceutical disintegration apparatus. Disintegration times were normalized by the film thickness. 800 mL of buffer solution was added to a glass beaker and was equilibrated to 37° C. in a water bath. A standard pharmaceutical disintegration carousel was loaded with dry film samples sandwiched between two plastic tubes, where the film was open to the environment on both sides. A stainless-steel ball bearing was placed on top of each film to use as a visual indicator for when the film dissolved. Disintegration time was measured with a stopwatch and then normalized by film thickness. If films were not dissolved within 1 hour the test was stopped and recorded as 60 min, but a note was made that the films did not dissolve. The disintegration time was then multiplied by 0.74 mm to calculate disintegration times for a common thickness of capsule.

| | Disintegration | | Disintegration | Disintegration |
|---|---|---|---|---|
| Sample | time pH 1.2 | | time pH 6.8 | time pH 12 |
| Example 1a | 5.36 | min | 1.41 min | 3.57 min |
| Example 1b | 4.58 | min | 1.90 min | 2.18 min |
| Example 1c | 3.74 | min | 1.46 min | 1.38 min |
| Example 2a | 15.31 | min | 5.73 min | 4.78 min |
| Example 2b | 9.05 | min | 8.37 min | 5.92 min |
| Example 2c | 11.26 | min | 3.01 min | 1.78 min |
| Example 2d | 9.36 | min | 3.64 min | 3.60 min |
| Example 2e | 11.87 | min | 3.54 min | 3.60 min |
| Comp. example A | DND* | | 4.37 min | 3.19 min |
| Comp. example B | 4.18 | min | 3.19 min | 3.27 min |

*DND = did not dissolve

### Comparative Example C

Gels were prepared as in example 2, however a low ester pectin (37% esterification, 0% amidation) was used in place of the HE pectin.

| | Disintegration | | Disintegration | Disintegration |
|---|---|---|---|---|
| Sample | time pH 1.2 | | time pH 6.8 | time pH 12 |
| Example 1a | 5.36 | min | 1.41 min | 3.57 min |
| Example 1b | 4.58 | min | 1.90 min | 2.18 min |
| Example 1c | 3.74 | min | 1.46 min | 1.38 min |
| Example 2a | 15.31 | min | 5.73 min | 4.78 min |
| Example 2b | 9.05 | min | 8.37 min | 5.92 min |
| Example 2c | 11.26 | min | 3.01 min | 1.78 min |
| Example 2d | 9.36 | min | 3.64 min | 3.60 min |
| Example 2e | 11.87 | min | 3.54 min | 3.60 min |
| Comp. example A | DND* | | 4.37 min | 3.19 min |
| Comp. example B | 4.18 | min | 3.19 min | 3.27 min |
| Comp. example C | 37.89 | min | 4.04 min | 4.18 min |

### Example 3

### Preparation of Soft Capsules Comprising HE Pectin and Modified Corn Starch as the Film-Forming Polymers

A film-forming composition containing 20% HE pectin, 15% modified corn starch, 25% glycerine and 40% water was prepared under the same conditions as described in Example 2 in a 4-gallon Ross CDA4 mixer. Transfer pipes were preheated to 78° C. and spreader boxes were preheated to 82° C. The drums and dies were cooled throughout the run to maintain 20° C. The film thickness was adjusted to 0.74 mm and fed through the Farmateck FTK55 encapsulator. The wedge was then placed on the dies and films and heated until capsules sealed at around 55° C. Capsules were made using 7.5 oval dies with light mineral oil filled at 400 mg. The wet capsules were then placed on a tray and left in a low humidity room at 20° C. and 5-15% R.H. for 2 hours before being transferred to a drying oven at 40° C. and left overnight. Dried capsules were then removed from the oven to cool, then stored in sealed plastic bags until ready for testing.

Capsule physical properties were tested using a TA.XT plus texture analyzer equipped with a 2 mm probe operating at 0.5 mm/sec (rupture force), or a 38 mm probe operating at 5 mm/sec (burst strength). Capsules were tested with the seams parallel to the probe and the peak force to rupture the capsule or have the capsule burst at the seam was recorded.

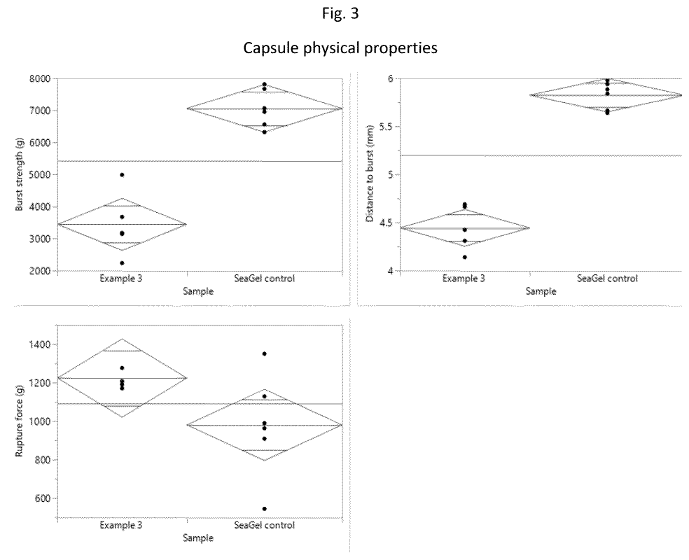

The results are shown in FIG. 3, from which it appears that capsules according to Example 3 show an equivalent hardness (determined as the force required to rupture the capsules) to capsules comprising carrageenan, whereas the burst strength and elasticity (distance to burst) is slightly lower. Nevertheless, the burst strength and elasticity determined for the capsules of this specification are considered sufficient to make them a commercially viable option.

All capsules dissolved in pH 1.2 media in 7.6 min (average of 5 capsules). In pH 6.8 media, the capsules dissolved in 8.7 min (average of 4 capsules) which is within the immediate release requirement of less than 15 minutes.

### Example 4

### Preparation of Hard Capsules Comprising HE Pectin as the Film-Forming Polymer

Hard capsules were prepared using a five-pin dipping system. The dipping solution was prepared by adding 20 g of HE pectin to 80 g of DI water that was pre-heated to 50° C. The HE pectin solution was then stirred with an overhead stirrer equipped with a propeller blade for 2 hours. The warm solution was then transferred to a pre-warmed aluminum trough for the capsule dipping. The cold pins were dipped into the warm solution and held for 10 seconds. After the pins were removed from the solution, they were oscillated 50 times to evenly distribute the solution. Once the oscillations were complete, the pins came to rest in an upright position and the capsules were dried in place for 16 hours before being removed.

### Example 5

### Film Coated Tablets

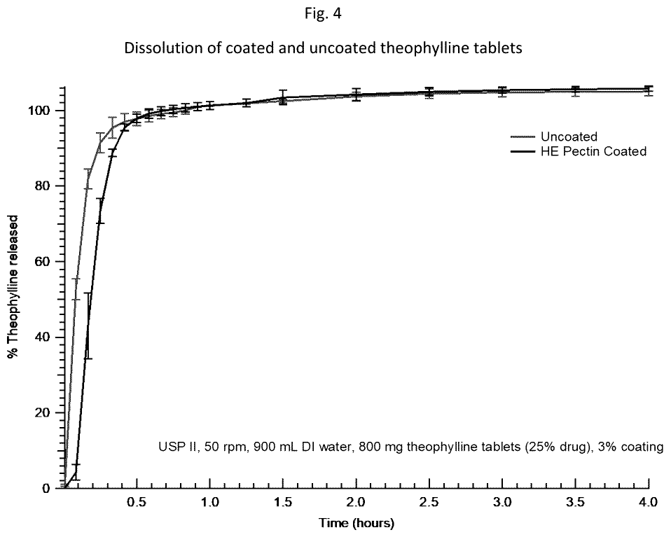

Coated tablets were prepared using a Vector laboratory development coating system. A 5% aqueous pectin solution was prepared by adding the high ester pectin and FD&C red 40 dye to pre-heated DI water. The solution was then mixed via an overhead stirrer for 2 hours and stored until needed. 550 g of pre-prepared 800 mg theophylline tablets were placed into the pan coater rotating at 20 rpm. The pectin solution was sprayed at 2.5 g/min with an inlet air temperature of ˜65° C. Tablets were coated with 3% weight gain and then drug dissolution testing was performed to evaluate effectiveness of the coating. The results are shown in FIG. 4, from which it appears that the release of theophylline from coated tablets is very similar to release from uncoated tablets, i.e. the HE pectin coating provides for immediate release of the active ingredient.

1. A film-forming composition comprising high ester (HE) pectin as a film-forming polymer and water.

2. The composition of claim 1, wherein the HE pectin exhibits a degree of esterification higher than 52%.

3. The composition of claim 1, wherein the HE pectin exhibits a degree of amidation of 0%.

4. The composition of claim 1, comprising HE pectin in an amount of 10-35% by weight of the composition.

5. The composition according to claim 1, further comprising one or more buffering agents.

6. The composition of claim 1, further comprising a second film-forming polymer selected from the group consisting of a native or chemically modified starch, xanthan gum, sodium alginate, β-1,3-glucan and carrageenan.

7. The composition of claim 6, wherein the starch contains less than 100% amylopectin.

8. The composition of claim 6, wherein the second film-forming polymer is hydroxypropylated starch.

9. The composition of 16, wherein the plasticizer is selected from the group consisting of glycerol, sorbitol, maltitol, polydextrose, polyethylene glycol and mixtures thereof.

10. The composition of claim 1, further comprising a divalent cation salt.

11. A capsule comprising a capsule shell prepared from the film-forming composition of claim 1 and a fill material.

12. The capsule of claim 11, wherein the fill material comprises a pharmaceutically active ingredient, dietary supplement, flavouring agent, foodstuff, agrochemical or scent.

13. The capsule of claim 11, wherein the fill material is a liquid, semi-solid, or solid material.

14. An immediate release capsule, wherein the immediate release capsule comprises:
a film-forming composition of claim 1;
a fill material; and
a pharmaceutically active ingredient, dietary supplement, flavouring agent, foodstuff, agrochemical or scent included in the fill material.

15. A method of forming a coated dosage form, the method comprising coating a dosage form with a film-forming composition of claim 1.

16. A film-forming composition of claim 1, wherein the composition further comprises a plasticizer.

17. The composition according to claim 5, wherein the one or more buffering agents is/are selected from the group consisting of sodium citrate, sodium potassium tartrate and sodium tripolyphosphate.

18. The composition of claim 6, wherein the second film-forming polymer is selected from the group consisting of corn starch, rice starch, potato starch, pea starch, tapioca starch wheat starch, rye starch, oat starch, barley starch and starch from pulses and mixtures thereof.

19. The composition of claim 6, wherein the second film-forming polymer is hydroxyethylated starch.

20. The composition of claim 10, wherein the divalent cation salt is CaCO₃.
